(19) United States
(12) Patent Application Publication
(10) Pub. No.: US 2008/0004566 A1
(43) Pub. Date: Jan. 3, 2008

(54) Gastrointestinal insufflation device and method

(76) Inventor: Dale Sloan, Fort Wayne, IN (US)

Correspondence Address: FROST BROWN TODD, LLC, 2200 PNC CENTER, 201 E. FIFTH STREET, CINCINNATI, OH, 45202, US

(21) Appl. No.: 11/479,546

(22) Filed: Jun. 30, 2006

(51) Int. Cl. A61M 1/00

(52) U.S. Cl. 604/30

(57) ABSTRACT

Disclosed is an insufflation device and method operably configured to reduce the number of false positive and false negative tests associated with checking the integrity of an anastomotic connection. Versions include a valve assembly configured to facilitate the delivery of pressure via a catheter to an anastomotic site without having to remove an associated pressure source.

### FIELD OF THE INVENTION

The present invention relates, in general, to medical insufflation devices and methods and, in particular, to testing anastomotic connections for integrity.

### BACKGROUND OF THE INVENTION

In the field of general surgery, circumstances arise where it is necessary to divide and reconnect portions of the gastrointestinal tract. After dividing a portion of the gastrointestinal tract, which may include any region from the esophagus to the rectum, a portion of the tract is generally removed due to a medical condition. A plurality of reasons exist for such a procedure including, for example, rearranging the intestinal anatomy, such as is common in bariatric or weight loss surgery, neoplasia, diverticular disease, and inflammatory bowel disease.

Following the removal or rearranging of tissue, it is generally necessary to restore the integrity of the patient's gastrointestinal tract such that normal function may be regained. Typically, this is accomplished by creating an anastomotic connection between the divided tissue sections. However, when the intestine is reconnected or anastomosed, there is the potential for a portion of the anastomosis to be incomplete or inadequate. This can lead to an anastomotic leaking of intestinal contents, possibly resulting in abscess formation, peritonitis, and even death.

Because of the potentially serious nature of an anastomotic leak, it is advantageous to check the integrity of the anastomotic connection. Such a check is commonly done by forcing gas, such as air or oxygen, through a tube or catheter into the inner lumen of the gastrointestinal tract in the area of the anastomosis. The portion of the tract being tested is distended with the gas using adequate pressure to cause the gas to leak out through any defects present.

While a portion of the tract is being insufflated with gas or air, it may also be submerged beneath a water or saline solution such that any leaking gas may be identified by bubbles in the liquid. Should bubbling occur, a surgeon will be alerted to the presence and possibly the location of the defect from the location of the bubbling.

Currently, insufflation of a patient's gastrointestinal tract usually involves placing a catheter into the lumen of the tract near the anastomosis site. When the anastomosis is in the esophagus, stomach, or proximal small bowel, the catheter is generally placed through the nose or mouth, then down the esophagus into the proper position. A large syringe filled with gas may then be used to force gas through an associated catheter.

If the anastomosis is in the rectum, it is usually tested by placing a rigid sigmoidoscope through the anus and into the rectum, where gas is then forced through the sigmoidoscope. This is often accomplished using a compressible pump with a one-way valve attached to the side of the sigmoidoscope.

In both of these methods of anastomotic testing, the volume of gas forced into the gastrointestinal tract is variable and is determined by the person compressing the gas. Such a method of control may result in pressure that is inadequate to force gas through a defect, causing a false negative. Alternatively, the volume of gas and the intraluminal pressure may be excessive, which could result in trauma to the intestinal wall, damage to the anastomotic connection, and/or forcing gas through an adequate suture or staple line causing a false positive.

Appropriate intraluminal pressure can be difficult to determine, even for experienced personnel. Often, however, the individual placing gas through the catheter or sigmoidoscope may have little experience with the technique and may tend to over or under insufflate.

It would therefore be advantageous to provide a device and method for testing the integrity of an anastomotic connection. It would be further advantageous to provide a device and method for testing the integrity of anastomotic connections that reduces the occurrence of false negative or false positive results. It would be advantageous to provide a device and method for testing the integrity of an anastomotic connection that reduces the likelihood that an anastomotic connection or a patient's gastrointestinal tract will be harmed. It would also be advantageous to provide a device and method for testing the integrity of anastomotic connections that is efficient and/or cost effective.

### DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a perspective view of one version of an insufflation device having a fluid source, a valve assembly, and a delivery member;

FIG. 2 is a right side cross-sectional view of one version of the valve assembly of FIG. 1 shown with the fluid source retracted to draw air therein;

FIG. 3 is a right side cross-sectional view of the valve assembly of FIG. 2 shown with the fluid source pushed forward such that air is driven through the valve assembly;

FIG. 4 is a right side cross-sectional view of the valve assembly of FIG. 2 shown with a pressure valve open after the internal pressure of the valve assembly exceeds a threshold;

FIG. 5 is a right side cross-sectional view of an alternate version of the valve assembly of FIG. 1 shown with the fluid source retracted to draw air therein;

FIG. 6 is a right side cross-sectional view of the valve assembly of FIG. 5 shown with the fluid source pushed forward such that air is driven through the valve assembly;

FIG. 7 is a right side cross-sectional view of the valve assembly of FIG. 5 shown with a pressure valve open after the internal pressure of the valve assembly exceeds a threshold;

FIG. 8 is a perspective view of the insufflation device of FIG. 1 shown inserted into a patient's rectum;

FIG. 9 is a flow chart depicting one method for testing an anastomotic connection; and

FIG. 10 is a flow chart depicting a method for testing an anastomotic connection.

### DETAILED DESCRIPTION OF THE INVENTION

Disclosed are versions of an insufflation device that may reduce the number of false positives and false negatives associated with testing the integrity of an anastomotic connection. Versions disclosed herein may also reduce the likelihood of damaging a patient's gastrointestinal tract and/or an anastomotic connection. Versions disclosed herein may also increase the continuity of an anastomotic testing procedure by allowing a syringe or the like to be used substantially continuously without having to repeatedly remove and reload the syringe during a procedure.

A false negative, for purposes of versions contained herein, refers to an anastomotic test that improperly suggests to a clinician that an anastomotic connection is free of defects. This may result, for example, when insufficient insufflation is provided to the region of a defective anastomosis to penetrate the bowel wall even though a true defect exists. A false positive, for purposes of versions contained herein, refers to an anastomotic test that improperly suggests to a clinician that an anastomotic connection contains a defect. This may result, for example, when the patient's bowel is over insufflated such that a suitable suture or staple line is penetrated by the pressurized gas or the like.

For purposes herein, the term fluid shall be defined as an amorphous substance whose molecules may move past one another such as a liquid or gas. Fluids include, for example, water, saline, oxygen, air, dye, gasses, and liquids. The term plenum shall be defined as a condition, space, or enclosure in which fluid, air, and/or gas is at a pressure greater than that of the outside atmosphere for a period of time. The plenum may further include a condition, space, or enclosure in which fluid, air, and/or gas is at atmospheric pressure and/or is at a pressure less than that of the outside atmosphere for a period of time.

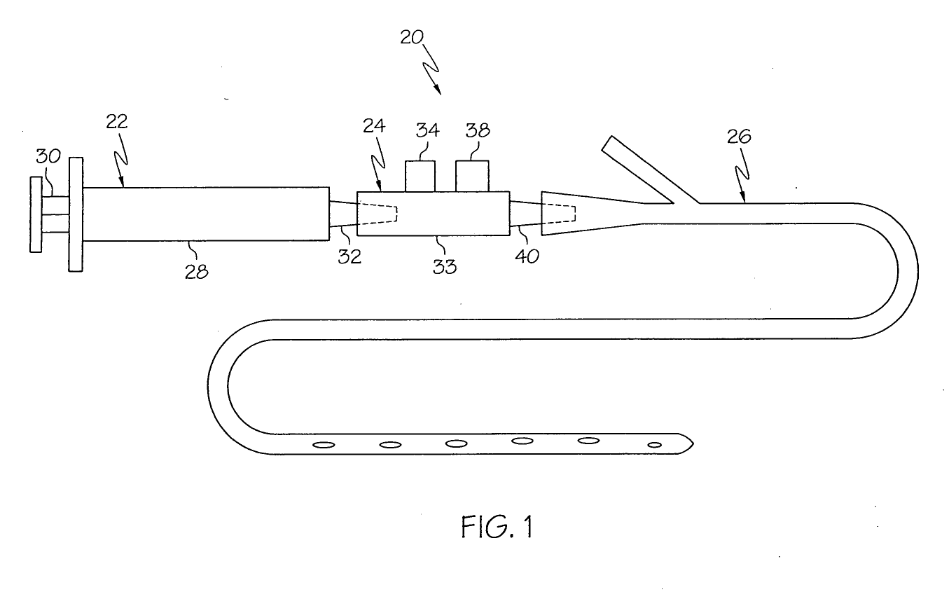

Referring to FIG. 1, one version of an insufflation device 20 is disclosed including a fluid source 22, a valve assembly 24, and a catheter 26. The fluid source 22 may be any suitable device, mechanism, or component for delivering fluid, oxygen, gas, dye, and/or air to the valve assembly 24. The fluid source 22 may be, for example, a 50 cc to 60 cc syringe having a body 28 with a retractable plunger 30 operably configured to translate therein such that a pressure differential is created. The fluid source 22 may be provided with an adapter 32 configured to mate the fluid source 22 with the valve assembly 24. The adapter 32 may be operably configured to fluidly connect the fluid source 22 and the valve assembly 24 such that a pressure differential created in the fluid source 22 is translated to the valve assembly 24. It will be appreciated that the fluid source 22 may be any suitable device for delivering a positive pressure through the valve assembly 24 including, for example, a low pressure pump providing continuous fluid or gas delivery or a bulb type pump. The valve assembly will be discussed in further detail herein.

The catheter 26 may be any suitable catheter, for example, an off-the-shelf catheter such as a 12 mm diameter catheter about three feet in length. Providing an insufflation device 20 that incorporates off-the-shelf components, such as a syringe and a catheter, with a single valve assembly may reduce the cost and complexity of testing an anastomotic connection. For example, the valve assembly may be adapted to accept a wide range of syringes and catheters such that the insufflation device 20 may be constructed by a clinician easily and cost-effectively from readily available components.

Still referring to FIGS. 1-4, one version of the valve assembly 24 includes a body 33, a first valve 34, a second valve 36, and a third valve 38. The body 33 may be for example, machined or injection molded polycarbonate and may be cofigured such that it may be sterilized and/or disposable. The body 33 may have a proximal end configured to mate with the fluid source 22 and a distal end configured to mate with the catheter 26, although other suitable configurations are contemplated. The first and second valves 34, 36 may be any suitable valve such as, for example, a flap-type valve or a bivalve. The first valve 34 may be an intake valve operably configured to allow the intake of atmospheric air, oxygen, gas, and/or fluid into the chamber defined by the body 33 when a negative pressure differential is created by, for example, retracting the retractable plunger 30 of the fluid source 22. The first valve may be, for example, a 2 psi check valve. The first valve 34 may be configured such that upon creation of a positive pressure differential within the body 33, the first valve 34 is closed, thereby prohibiting the gas or the like from exiting the body 33.

The first valve 34 may also be connected, for example, to an exterior oxygen, gas, air, pressure, dye, and/or fluid source for the delivery of the like to the insufflation device 20. In a further version, where the fluid source 22 includes continuous delivery of a fluid or gas from a low pressure pump or the like, it will be appreciated that the valve assembly 24 may not be provided with a first valve 34 or, if present, that the first valve 34 may remain closed for the duration of the testing procedure. The first valve 34 may be laterally connected to the body 33 or may be otherwise suitably configured.

Referring to FIGS. 1-4, one version of the second valve 36 is depicted housed within the body 33 of the valve assembly 24. The second valve 36 may be an exit valve operably configured to allow air, gas, fluid, dye, or the like with a positive pressure to pass therethrough into the delivery member or catheter 26. The second valve 36 may, for example, be a 0 psi check valve. In one version, where the insufflation device 20 includes a syringe, the second valve 36 may close as fluid or the like is drawn into the body 33 and then into the syringe. As the retractable plunger 30 is depressed, the positive pressure created within the body 33 may close the first valve 34 and may open the second valve 36. The second valve may, in one version, be concentric with the body 33 and may be located distal to the first valve 34. It will be appreciated, in the version of the insufflation device 20 having a continuous fluid source, that the second valve 36 may remain open so long as the continuous fluid source is active.

Providing a first valve 34 and a second valve 36 configured in accordance with versions herein may comprise a two-way valve system that facilitates a more continuous delivery of pressure to an anastomotic site. For example, providing an insufflation device 10 with a syringe as the fluid source 22 may combine the benefits of mobility and ease of use of a syringe with a more continuous pressure delivery than that used in traditional insufflation systems. Combining the syringe with the first valve 34 and the second valve 36 may allow a physician to repeatedly retract and depress the retractable plunger 30 to deliver pressure without having to fully remove the syringe from the insufflation device. Versions combining the first valve 34, second valve 36, and a syringe may allow for the syringe or fluid source 22 to be permanently affixed to the valve assembly 24 for the duration of the procedure, where the insufflation device 20, or portions thereof, including a syringe and a valve assembly 24, may be manufactured as a single reusable or diposable component.

Still referring to FIGS. 1-4, the valve assembly 24 may further include a third valve 38 that may be a pressure valve. In one version, the third valve 38 may be biased in a closed position adjacent the body 33 and may be operably configured open to release positive pressure that exceeds a threshold within the body 33. In one version, the third valve 38 may have a pre-determined threshold, where any pressure within the body 33 in excess of the threshold will open the third valve 38 to the atmosphere, thereby dissipating the internal pressure of the valve assembly 24 back to an acceptable level. The third valve may, for example, be a 1 psi check valve, a 0.5 psi to 1.0 psi check valve, a 0.7 psi to 0.8 psi check valve, or any other suitable valve. The third valve 38 may be positioned distal to the second valve 36 such that when positive pressure is being delivered through the valve assembly 24, it may be maintained at a desirable level. In versions of the valve assembly 24, the third valve 38 may be provided with a single pre-determined threshold, multiple pre-determined thresholds, and/or a variable threshold that may be set by a clinician for a particular procedure.

In versions herein, the third valve 38 may be operably configured with an indicator 39 to indicate to the clinician when the pressure threshold has been exceeded or reached. The indicator 39 may be the third valve 38, where the third valve 38 may be operably configured to release air pressure above the threshold at a decibel level sufficient for the clinician to hear that the third valve 38 has been opened. The insufflation device 20 and/or the third valve 38 may be provided with an aural, visual, electrical, and/or mechanical indicator operably configured to inform the clinician that the threshold has been reached and/or exceeded. The indicator 39 may be, for example, a bourdon tube or a pressure gauge. It is further contemplated that the insufflation device 20 may be provided with pressure sensors such that a clinician may monitor, in real-time or substantially real-time, the pressure of insufflation device 20. It is further contemplated that the insufflation device 20 may be provided with a manual or automatic feedback loop such that pressure may be delivered until the pre-determined threshold is met and/or exceeded.

By including an indicator 39, which may indicate when the pre-determined threshold has been met and/or exceeded, the clinician may be able to readily establish when a suitable level of pressure has been administered to the anastomotic site. For example, when a clinician hears gas escaping from the third valve 38, the clinician will know that the level of pressure being administered is sufficient. Additionally, due to the setting of the pre-determined threshold, the clinician may have an estimate as to the level of pressure administered. It will be appreciated that more sophisticated devices involving, for example, pressure sensors, visual indicators, aural indicators, and/or combinations thereof are consistent with versions herein and may be incorporated, for example, depending upon the delicacy or specific requirements of a particular procedure.

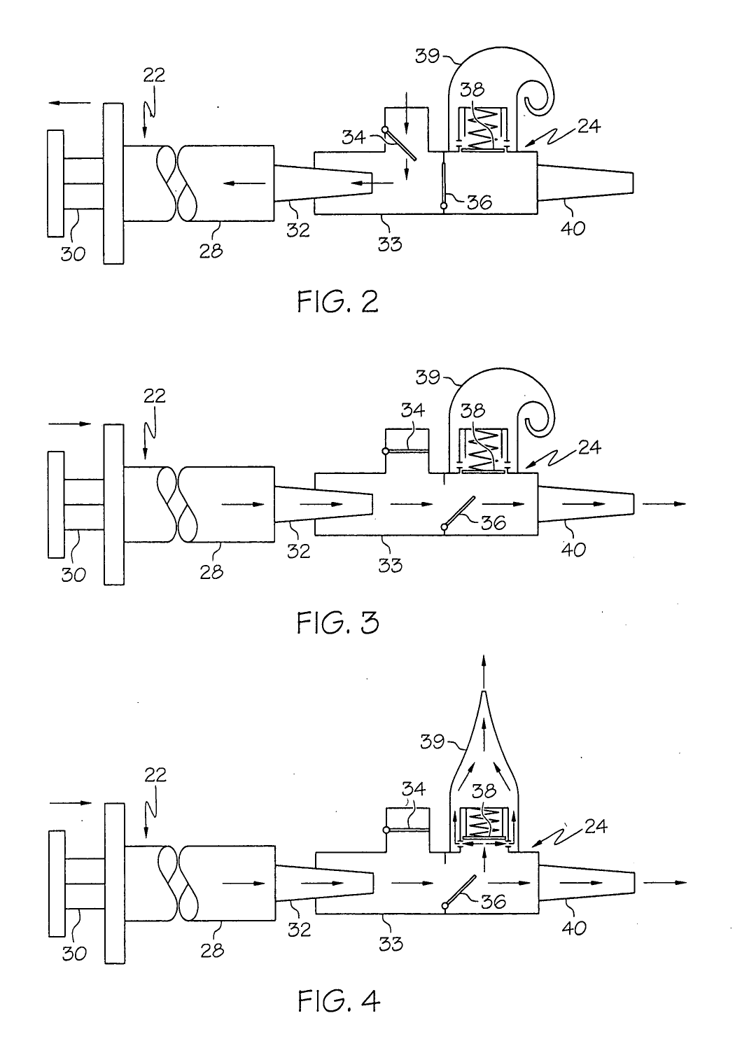

In a further version, as shown in FIGS. 2-4, the third valve 38 or any other suitable component of the insufflation device 20 may be provided with an indicator 39 in the form of a bourdon tube or any other inflatable pressure testing device. A bourdon tube may be initially coiled into a circular arc or formed into a helix of several turns such that, as pressure is applied to the tube, the oval section tends to round out becoming more circular in section. The inner and outer arc lengths may remain approximately equal to their original lengths, where the only recourse for the tube is to uncoil. When using an insufflation device having an indicator 39 in the form of a bourdon tube or the like, the clinician may visually be able to see the tube uncoil indicating that the third valve 38 has been opened. Additionally, it is contemplated that the indicator 39, such as a bourdon tube, may be provided in the absence of a third valve 38, where the bourdon tube acts as the sole indicator and guide for the delivery of a desired level of fluid pressure. It is further contemplated that the bourdon tube or inflatable indicator may be provided with a noise-making feature such that the inflation of the tube creates a visual indicator in addition to an aural indicator.

Providing the third valve 38 may reduce both the number of false negative and false positive anastomotic tests. For example, the number of false positive tests may diminish because the third valve 38 may be set to open to the atmosphere at a threshold less than that needed to create a false positive response and/or to damage a patient's gastrointestinal tract. In one version, a high level of pressure may be delivered from the fluid source 22, where the valve assembly 24 and the third valve 38 may then reduce the delivered pressure to a suitable level for transmission to the catheter 26. The reduction of pressure by the valve assembly 24 to a desirable level, irrespective of the level of pressure introduced, may reduce false positive test results and the occurrence of damage to a patient's gastrointestinal tract, and may make the insufflation device 20 easier to operate by an inexperienced user.

Additionally, false negative test results may also be reduced by the incorporation of the third valve 38. In manually delivering pressure, for example, with the use of a syringe, the user of prior systems may under insufflate an anastomotic site for fear of delivering too much pressure that could damage the anastomotic site or create a false positive test result. Providing a third valve 38 in the form of a pressure release valve may assure users that they can deliver relatively high amounts of pressure without causing damage to an anastomotic site. In practice, if users are instructed to depress the retractable plunger 30 or otherwise deliver pressure until the third valve 38 opens, then a reasonably consistent level of pressure at a relatively calculable level may be administered. Such a device and method may improve the overall quality of anastomotic testing by maintaining patient safety and increasing accuracy.

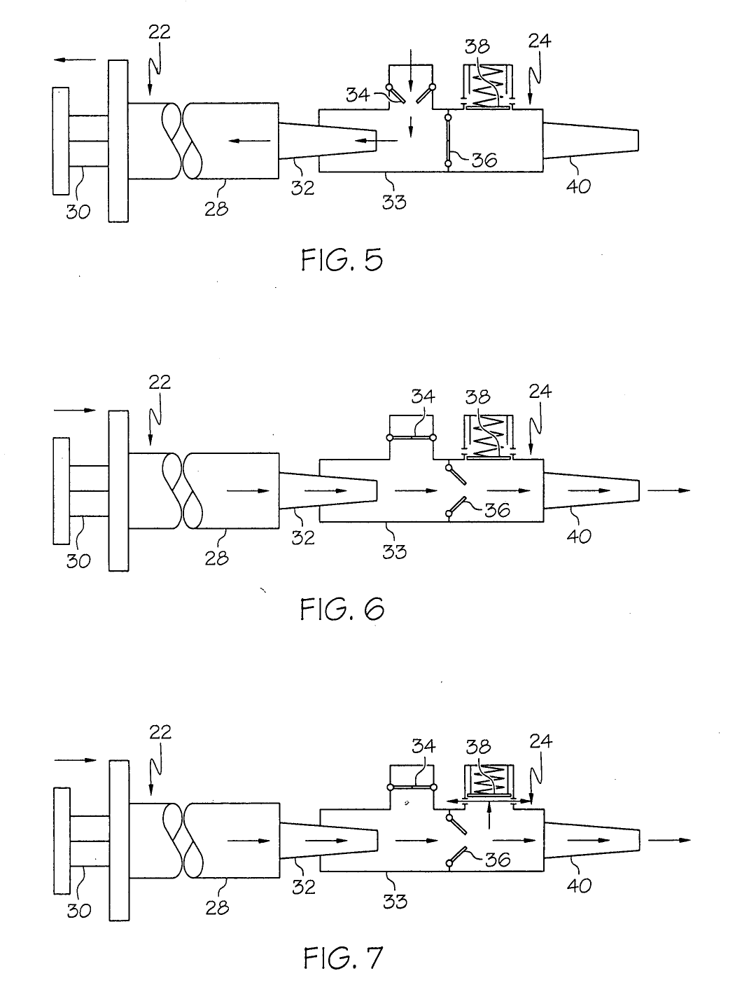

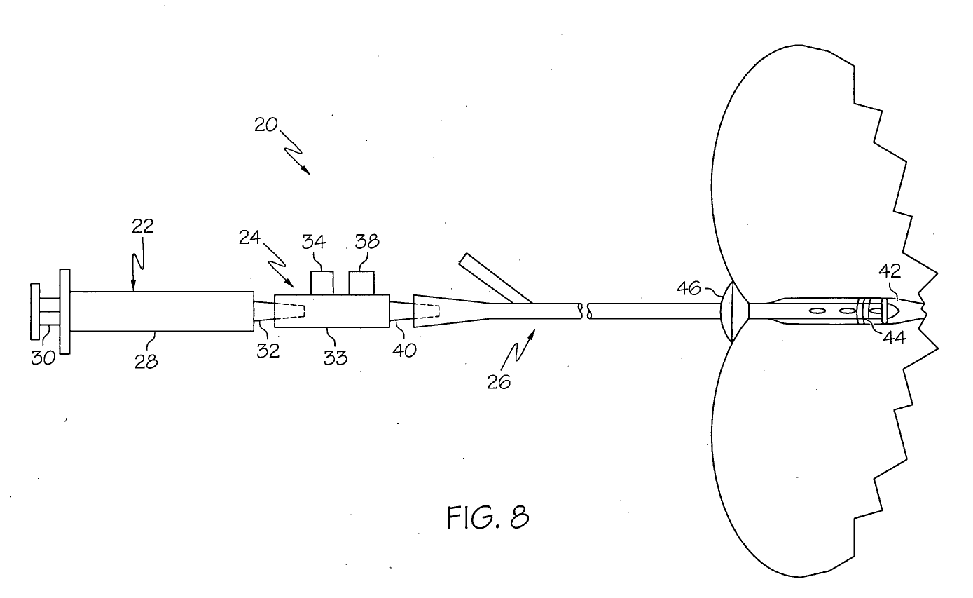

Referring to FIGS. 5-7, disclosed is an alternate version of the insufflation device, where the first valve 34 and the second valve 36 are bivalve-type valves. It will be appreciated that the insufflation device 20 may be provided with any suitable number of valves in any suitable configuration. The insufflation device 20 may include one or a plurality of first valves 34, one or a plurality of second valves 36, and/or one or a plurality of third valves 38. The insufflation device may include any suitable pressure delivery or valve system operated manually, mechanically, hydraulically, pneumatically, and/or electrically. The valve assembly 24 may include an adapter 40 operably configured to mate with a catheter 26 or any other suitable component suitable for anastomotic testing. The valve assembly 24 may include any suitable type of valve including, but not limited to, stop valves, globe valves, gate valves, butterfly valves, solenoids, flap-type valves, and/or bivalves. The fluid source 22, the valve assembly 24, and the catheter 26 may be constructed from any suitable material such as, for example, metal, rubber, synthetics and/or medical grade plastics.

Referring to FIG. 8, one version of the insufflation device 20 is shown inserted into a patient's rectum 42 to test the integrity of an anastomosis 44. The catheter 26 may include a plug 46 or any other suitable device by which the dispersion of pressurized gas is reduced. The insufflation device 20 may be provided with a seal, clamp, or other suitable closure device (not shown) near or at the distal end of the catheter 26 such that the rectum may be substantially sealed for testing the anastomosis 44. It will be appreciated that any suitable seal, clamp, or closure device may be used in conjunction with the catheter 26 and/or the insufflation device 20 such that desirable regions may be substantially sealed to reduce dispersion of air or the like in an anastomotic region.

The illustrated version of the insufflation device 20 of FIG. 8 is disclosed by way of example and is not intended to limit the present invention. It will be appreciated that the insufflation device 20 may be used with any portion of the gastrointestinal tract, including the upper and lower gastrointestinal tracts. Additionally, it will be appreciated that the insufflation device 10 may be used for veterinary purposes in addition to human medical purposes. It will be further appreciated that the insufflation device 20 may be used for any other suitable procedure, including those where an anastomotic site is not being tested, such as for the distention and irrigation of a vein, where the delivery of fluid, air, oxygen, dye, liquid, and/or combinations thereof is advantageously delivered at a pressure below a threshold and/or at a substantially consistent level.

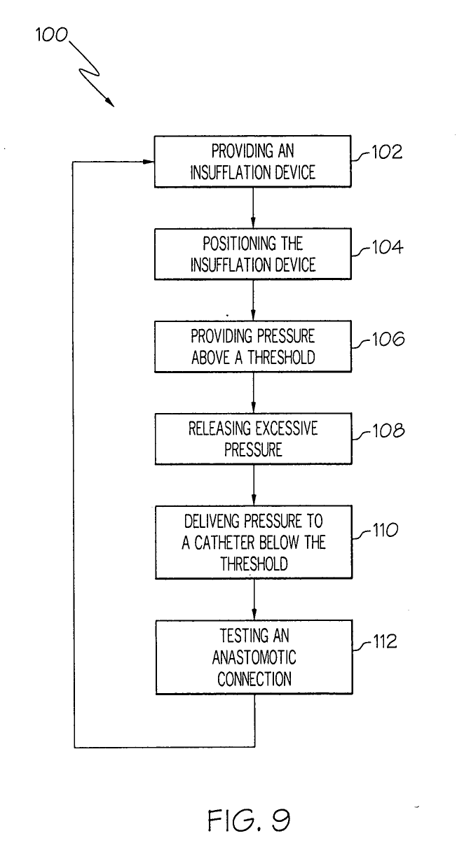

Referring to FIG. 9, disclosed is one version of a method 100 for using an insufflation device, such as insufflation device 20, to test an anastomotic connection. Step 102 of the method 100 includes providing an insufflation device, which may be insufflation device 20 or any other suitable insufflation device. The insufflation device may include a fluid source, a valve assembly, a catheter, a sigmoidoscope, or any other suitable component. The insufflation device may provide continuous, substantially continuous, or intermittent delivery of pressure to an anastomotic site.

Step 104 includes placing the insufflation device adjacent the region of the anastomotic connection such that the integrity of the anastomotic connection may be tested. The region of the anastomotic connection may be anywhere in a patient's gastrointestinal tract including the upper gastrointestinal tract and lower gastrointestinal tract. The positioning of the insufflation device step 104 may include positioning a catheter, a sigmoidoscope, or any other suitable component.

Step 106 includes providing pressure above a threshold. The pressure may be provided by a fluid source 22, or by any other suitable pressure delivery device. The pressure delivered by the fluid source 22 may be continuous, substantially continuous, or intermittent. The pressure delivered by the fluid source 22 may also be ramped up or ramped down. The fluid source 22 may, for example, be a syringe, a low pressure pump, or any other suitable device. The threshold may be established as a level of pressure which is sufficient to accurately test the integrity of an anastomotic connection, but is low enough such that pressure below the threshold does not result in false positive anastomotic tests and does not damage a patient's gastrointestinal tract. The threshold may be established by the choice or tuning of, for example, a rate limiting orifice and/or a third valve 38 that is operably configured to release air pressure or the like into the atmosphere above a pre-determined level.

The threshold may be modified depending on the needs or location of a particular anastomotic connection. The threshold may be modified by, for example, using an insufflation device having a different pre-established threshold, changing the third valve 38 of an insufflation device to a valve having a different threshold, and/or manually tuning a valve operably configured to allow a clinician to adjust the threshold.

Step 106 further includes providing air, gas, fluid, dye, and/or oxygen pressure from a fluid source at a level above the pre-established threshold. The pressure delivered, at least initially, above a pre-established threshold may be provided by, for example, a syringe operated by a user or by a low pressure pump.

Step 108 includes releasing excessive pressure from the insufflation device. Excessive pressure is defined as the pressure within, for example, the valve assembly 24 that exceeds the pre-established threshold. Pressure above the threshold may open the third valve 38, which may be biased closed, such that excess pressure is released into the atmosphere. After the excessive pressure has been released, the third valve 38 may return to the closed position until the pressure provided again exceeds the pre-determined threshold.

Step 110 includes delivering pressure to a delivery member, such as the catheter 26, below the pre-established threshold. In one version, the third valve 38 may be operably configured such that excessive pressure is expelled into the atmosphere before reaching the catheter 26 and the anastomotic site. Because excessive pressure may be released by the third valve 38 in the valve assembly, the pressure delivered to the catheter 26 may be below the pre-determined threshold and therefore may be less likely to result in a false positive anastomotic test or to do damage to a patient's gastrointestinal tract.

Step 112 includes testing an anastomotic connection. The testing of an anastomotic connection step 112, in one version, includes delivering pressure through a catheter 26, or any other suitable delivery member, to an anastomotic region such that the fluid, air, liquid, oxygen, gas, and/or dye pressurizes the region of the anastomosis and, if a defect is present, will pass through the defect and indicate the defect to the clinician. By providing Steps 106 and 108, the method 100 may deliver a level of pressure that is low enough so as to not cause gastrointestinal damage or to cause false positive anastomotic tests. Additionally, the method 100 may reduce the number of false negative tests by allowing a user to deliver a relatively high level of pressure, more than the user might have delivered without the presence of a third valve 38, without fear of causing damage to a patient. By delivering a pressure until, for example, the third valve 38 releases the excess into the atmosphere, a clinician may more accurately gauge the approximate level of pressure being applied to an anastomotic site. Such a method may improve the quality and accuracy of anastomotic tests for both novice and experienced clinicians.

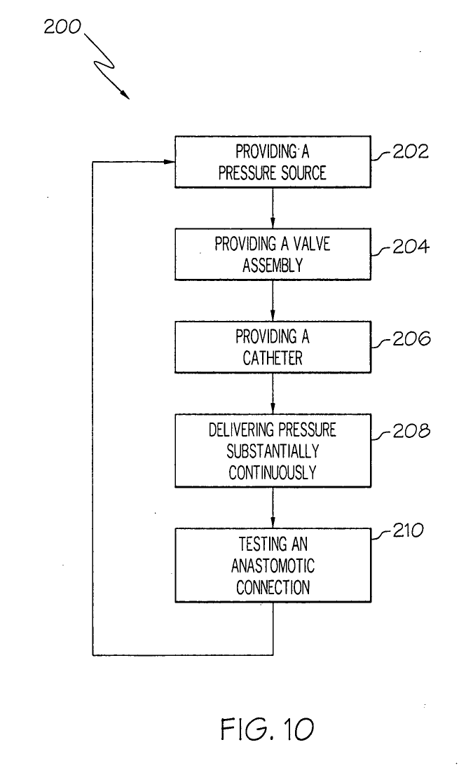

Referring to FIG. 10, disclosed is one method 200 for operating an insufflation device, such as insufflation device 20, such that substantially continuous pressure may be delivered to an anastomotic site for testing the integrity thereof. For purposes of this version, substantially continuous pressure refers to the use of a syringe or other fluid source operated by a user to deliver pressure to an anastomotic site where, in accordance with the method 200, the fluid source does not need to be disconnected or otherwise removed from the insufflation device to continue delivering pressure. For purposes of this version, although there will be lapses in pressure delivery when, for example, the retractable plunger 30 is retracted, the pressure delivery is still relatively substantially continuous because the syringe need not be removed, reloaded, and then reattached to continue delivering pressure to the anastomotic site.

Step 202 of the method 200 includes providing a fluid source, such as a syringe or other suitable fluid source 22, operably configured to deliver pressure to an anastomotic site. In the version of method 200 where the fluid source is a syringe, the pressure may be provided to the anastomotic site by depressing the plunger 30 of the syringe to create a positive pressure differential. When the plunger 30 has been fully depressed, it may be necessary to retract and reload the plunger before again depressing the plunger 30 to deliver pressure.

Step 204 includes providing a valve assembly, where the valve assembly may include an intake valve and an exit valve operably configured to allow for the substantially continuous delivery of pressure. For example, the valve assembly may be valve assembly 24, the intake valve may be the first valve 34, and the exit valve may be the second valve 36. When the plunger 30 is retracted to load the syringe or fluid source 22, the intake valve may open due to the negative pressure created within the valve assembly such that air or the like is drawn into the syringe or fluid source 22. After the plunger 30 has been partially or fully retracted, the intake valve may close due to a bias to close when negative pressure is not present.

As the plunger is depressed, the exit valve, such as second valve 36, may open allowing the positive pressure to pass through the valve assembly. The exit valve may be configured such that it is closed when the plunger is retracted and opened when the plunger is depressed.

Step 206 includes providing a delivery member, such as catheter 26, or any other suitable delivery component. The catheter, or the like, may be operably configured to deliver air, gas, fluid, and/or oxygen pressure passing through the valve assembly to an anastomotic site to test the integrity thereof.

Step 208 includes delivering pressure substantially continuously. By providing an intake valve and an exit valve, in accordance with Step 204, pressure may be delivered via a catheter or the like to an anastomotic site substantially continuously without removing the syringe or fluid source 22. For an anastomotic test requiring more pressure than can be delivered by depressing a syringe a single time, the method 200 allows a clinician to retract the plunger 30 to reload the syringe without having to remove the syringe to reload it. Such a two-way valve system may allow the clinician to use and load the syringe or fluid source 22 as many times as necessary without having to remove the syringe or the like for the duration of the procedure.

Step 210 includes testing an anastomotic connection. The anastomotic connection may be located anywhere in a patient's gastrointestinal tract or in any other suitable location. By providing substantially continuous pressure with a syringe or fluid source 22, the integrity of an anastomotic site may be tested more quickly, efficiently, and/or accurately. By not having to remove and reload the syringe after every use, the clinician may be able to provide a more consistent level of pressure to an anastomotic site to determine if any defects are present. Additionally, the method 200 may provide the benefits of substantially continuous pressure delivery with the mobility and ease of use associated with the use of a syringe as a fluid source 22.

It will be appreciated that method 100 and method 200 may be used in combination or independently from one another. It will be further appreciated that the steps of method 100 and method 200 are listed in the depicted order by way of example only, where any suitable combination of steps may be provided to test an anastomotic connection. It will be further appreciated that components used in conjunction with the methods 100, 200 are shown by way of example only, where any suitable components may be used in accordance with versions herein.

In summary, numerous benefits have been described which result from employing the concepts of the invention. While the present invention has been illustrated by the description of several embodiments and while the illustrative embodiments have been described in considerable detail, it is not the intention of the applicant to restrict or in any way limit the scope of the appended claims to such detail. The foregoing description of one or more embodiments of the invention has been presented for purposes of illustration and description. It is not intended to be exhaustive or to limit the invention to the precise form disclosed. Obvious modifications or variations are possible in light of the above teachings without departing from the invention. It should be understood that every structure described above has a function and such structure can be referred to as a means for performing that function. The one or more embodiments were chosen and described in order to best illustrate the principles of the invention and its practical application to thereby enable one of ordinary skill in the art to best utilize the invention in various embodiments and with various modifications as are suited to the particular use contemplated. Accordingly, it is intended that the invention be limited only by the spirit and scope of the appended claims.

What is claimed is:

1. An insufflation device comprising:
(a) a fluid source, said fluid source having a proximal end and a distal end, where said fluid source is operatively configured for the delivery of an insufflation fluid;
(b) a valve assembly, said valve assembly including a body having a proximal end and a distal end, where said valve assembly includes;
(i) a first valve, where said first valve is operably configured to intake said insufflation fluid;
(ii) a second valve, where said second valve is operably configured to provide insufflation; and
(iii) a third valve, where said third valve is operably configured to maintain the pressure of said insufflation fluid below a threshold; and
(c) a member, where said member is associated with said valve assembly, where said member is operably configured to deliver fluid.

2. The insufflation device of claim 1, wherein said third valve includes an indicator.

3. The insufflation device of claim 2, wherein said indicator is a bourdon tube.

4. The insufflation device of claim 1, wherein the delivery of said insufflation fluid is intermittent.

5. The insufflation device of claim 1, wherein said fluid source is a syringe.

6. The insufflation device of claim 5, wherein said member is a catheter.

7. The insufflation device of claim 1, wherein said fluid source is selected from the group consisting of a syringe, a bulb-type pump, a low pressure pump, an intermittent fluid source, a continuous fluid source, an off-the-shelf fluid source, a mechanical fluid source, a pneumatic fluid source, a hydraulic fluid source, and combinations thereof.

8. The insufflation device of claim 1, wherein said valve assembly is operably configured to associate with one of a plurality of said fluid sources.

9. The insufflation device of claim 1, wherein said valve assembly is operably configured to associate with one of a plurality of said members.

10. A gastrointestinal insufflation device comprising:
(a) a pump, said pump comprising;
(i) a body, said body having a proximal end and a distal end;
(ii) an actuator associated with said body, where translating said actuator relative to said body creates a pressure differential;
(iii) an aperture, where said aperture is configured at the distal end of said body, where said aperture is operably configured for the input and output of air;
(b) a plenum, where said plenum is associated with said pump, where a negative pressure is created within said plenum at a first time and a positive pressure is created within said plenum at a second time;
(c) a delivery member, where said delivery member is associated with said plenum, where said delivery member is operably configured to deliver pressure to an anastomotic site to test the integrity thereof.

11. The gastrointestinal insufflation device of claim 10, wherein said pump is a syringe.

12. The gastrointestinal insufflation device of claim 10, wherein said plenum includes an intake and an output, where when said intake is in the open position, said output is in the closed position and where when said output is in the open position, said intake is in the closed position.

13. The gastrointestinal insufflation device of claim 10, wherein said plenum includes a pressure regulator.

14. The gastrointestinal insufflation device of claim 13, wherein said pressure regulator includes an indicator.

15. The gastrointestinal insufflation device of claim 10, wherein proximally translating said actuator creates said negative pressure in said plenum and distally translating said actuator creates said positive pressure in said plenum.

16. A method of testing a gastrointestinal anastomotic connection comprising:
(a) providing an off-the-shelf pressure source;
(b) providing a valve assembly comprising;
(i) a first valve, where said first valve is operably configured to intake insufflation fluid;
(ii) a second valve, where said second valve is operably configured to provide insufflation; and
(iii) a third valve, where said third valve is operably configured to maintain the pressure of said insufflation fluid below a threshold;
(c) providing an off-the-shelf delivery member;
(d) associating said off-the-shelf pressure source with said valve assembly;
(e) associating said valve assembly with said off-the-shelf delivery member; and
(f) performing an anastomotic test using the assembly of said off-the-shelf pressure source, said valve assembly, and said off-the-shelf delivery member.

17. The method of testing a gastrointestinal anastomotic connection of claim 16, wherein said off-the-shelf pressure source is a syringe.

18. The method of testing a gastrointestinal anastomotic connection of claim 16, wherein said off-the-shelf delivery member is a catheter.

19. The method of testing a gastrointestinal anastomotic connection of claim 16, wherein said third valve includes an indicator.

20. The method of testing a gastrointestinal anastomotic connection of claim 16, further comprising the steps of;
(g) creating a negative pressure within said valve assembly at a first time; and
(h) creating a positive pressure within said valve assembly at a second time.
